(19) United States
(12) Patent Application Publication
(10) Pub. No.: US 2014/0206055 A1
(43) Pub. Date: Jul. 24, 2014

(54) METHOD FOR VISCOSITY REDUCTION IN CO-FERMENTATION ETHANOL PROCESSES

(71) Applicant: Edeniq, Inc., Visalia, CA (US)

(72) Inventors: Kristoffer Ramos, Sanger, CA (US); Donna Santos, Visalia, CA (US); Padmavathy Desai, Fresno, CA (US); Prachand Shrestha, Visalia, CA (US); Richard Root Woods, Three Rivers, CA (US)

(73) Assignee: Edeniq, Inc., Visalia, CA (US)

(21) Appl. No.: 14/163,464

(22) Filed: Jan. 24, 2014

Related U.S. Application Data
(60) Provisional application No. 61/756,393, filed on Jan. 24, 2013.

(51) Int. Cl. C12P 7/10

(52) U.S. Cl. CPC C12P 7/10
USPC 435/165

(57) ABSTRACT

The present disclosure provides methods and compositions for reducing the viscosity of biomass process streams in an ethanol production process. The method comprises adding cellulase enzymes to a biomass feedstock that is fermented to produce ethanol, generating whole stillage and thin stillage streams from the post-fermentation biomass, and adding an additional enzyme or enzyme cocktail that reduces the viscosity of the whole stillage stream, thin stillage stream, concentrated thin stillage stream, and/or the syrup stream generated by evaporating the thin stillage.

### CROSS-REFERENCE TO RELATED APPLICATIONS

This application claims the benefit under 35 U.S.C. § 1.119(e) of U.S. Patent Application No. 61/756,393, filed Jan. 24, 2013, which is incorporated by reference herein in its entirety for all purposes

### TECHNICAL FIELD

This present invention relates to the conversion of biomass to biofuels and other products and co-products, including materials and methods for preprocessing, conversion, and post-processing the feedstock and post-processed residual materials from the biomass.

### BACKGROUND OF THE INVENTION

Corn is the most common feedstock for production of ethanol in the United States. Other feedstocks are used to a lesser degree—sugar beets, sugar cane, milo (sorghum), barley, corn stover, energy cane, and wood waste.

With respect to using starch-rich feedstock, such as corn, as the biomass, the kernels are made up of a variety of materials including starch, protein, oils, fiber, and various organic and inorganic compounds along with water. The endosperm, which contains mainly starch, typically accounts for approximately 80-85% (dry weight basis) of the corn kernel whereas the germ and the hull account for approximately 10-14% and 5-6%, respectively. The germ is high in oil, typically containing approximately 38-45% oil by weight.

Typically, the corn kernel contains between 68-75% starch, 10-12% fiber, 8-9% protein, 3-4% fat, and the balance being ash. More specifically, corn kernel fiber is a distinct portion of the overall corn kernel which can be defined as the cellulosic or fiber component. Corn kernel fiber contains roughly 33% lignin, 35% cellulose, and 32% hemicellulose, excluding bound starch, fat, and proteins in various amounts.

For conversion of corn and other starch-based biomass to ethanol the starch content is typically broken down or converted or hydrolyzed into sugars by enzymes, by unit operations also known as liquefaction and saccharification. The sugars are then fermented into ethanol by the metabolic action of yeast. Efficiency of starch conversion to sugar and ethanol varies from refinery to refinery and specific process to process. The corn fiber portion, which comprises additional polymeric sugar components or polysaccharides, is largely unconverted and remains as a portion of the residual solids in the post-fermented mash or beer.

Once fermentation has been completed, the beer is transferred through the beer well to the distillation system where solids and water are separated from ethanol through evaporation and filtration or other separation mechanisms. During distillation, ethanol product is evaporated from one stage and condensed in the next stage thereby concentrating the ethanol to approximately 95 vol %. The remainder of the water in the ethanol product is removed by molecular sieves or membrane concentration to achieve a product ethanol at greater than 99 vol %. The bulk water phase, also containing the soluble and insoluble solids, often referred to as whole stillage, is discharged from the bottom of the distillation column, passed downstream and further processed into co-products. These downstream processes can include various separation treatments such as centrifuging, evaporating, drying, filtering, extractions, and others.

The whole stillage consists of both suspended solids and dissolved solids of various ratios depending on the feedstock, preprocessing and fermentation conditions. In a typical dry mill corn ethanol facility the majority of the suspended solids are removed as roughly 35% wt solids wet cake, while the majority of the water with dissolved solids are split into recycled liquid stream or backset and thin stillage which is sent to an evaporator for concentration into a syrup stream. The evaporator concentrate of approximately 25 to 40% wt total solids can be mixed with the wet cake solids and either sold as a high moisture animal feed co-product or dried in a rotary or flash dryer to a 90 wt % solids powder known as distiller's dry grains with solubles (DDGS).

Depending on fermentation characteristics and the addition of various compounds and enzymes into slurry, liquefaction, and/or during simultaneous saccharification and fermentation (SSF), the characteristics of the syrup stream may be drastically affected, specifically (but not limited to) composition and rheology. This baseline process focuses on converting the primary starch based feedstock into glucose and the glucose into ethanol while allowing residual fibers to pass through the process and end up in the residual animal feed product even though these fibers have fermentable sugars components. This application describes an integrated process that utilizes the installed equipment and processing capacities and supports the conversion of the residual fibers and/or supplemental fibers into ethanol in a co-fermentation strategy without impacting the balance between the heat and water integration existing in the baseline facility or the efficiencies of these processes.

### BRIEF SUMMARY OF THE INVENTION

The present invention provides methods and enzyme compositions, specifically viscosity reducing enzymes (herein VREs), for managing the viscosity of the downstream process streams or stillage streams in an ethanol production plant that result from processing the starch based fermentation mash or whole corn mash slurries with cellulases and or other treatments to convert all or part of the component fiber into fermentable sugars. The process streams include whole stillage, thin stillage, concentrated thin stillage, and syrup streams.

In one aspect, a method for reducing the viscosity of process streams during production of ethanol from biomass is provided, the method comprising:

a. adding cellulase enzymes into a mash or feedstock comprising a mixture of a non-cellulosic sugar and a cellulosic sugar to co-produce non-cellulosic and cellulosic ethanol;

b. fermenting the mash to produce ethanol and a post-fermentation biomass;

c. generating process streams comprising whole stillage, thin stillage, concentrated thin stillage and/or syrup streams from the post fermentation biomass;

d. adding an additional enzyme or enzyme cocktail to at least one of the post fermentation process streams to reduce the viscosity of the syrup stream.

In some embodiments, the additional enzyme comprises xylanases, beta-glucosidases, and/or arabinofuranosidases or any combination thereof.

In some embodiments, the method further comprises introducing the additional enzyme or enzyme cocktail to the whole stillage, thin stillage, concentrated stillage, and/or syrup or any combination thereof.

In some embodiments, the additional enzyme or enzyme cocktail comprises one or more of the following: debranching enzymes, hemicellulases, pentosanases, xylanolytic enzymes, exoxylanases, endoxylanases, glucanases, exoglucanases, endo-beta-1,4-xylanases, exo-beta-1,4-xylosidase, alpha-L-rabinofuranosidase, endo-alpha-1,5-arabinanase, glucuronidases, alpha-glucuronidase, mannanases, endo-beta-1,4-mannanase, exo-beta-1,4-mannosidase, alpha-galactosidase, endo-galactanase, xylosidases, acetyl xylan esterases, glycosidases, beta-1,4-glycanases, pectinases, polygalactoronases, esterases, amylases, phytases, peroxidases, laccases, glucose oxidases, oxidoreductases, lipases, lipolytic enzyme, proteolytic enzymes, and/or proteases or any combination thereof.

In one embodiment, the cellulase enzymes are also added to the fermentation tank.

In some embodiments, the method further comprises mechanically pretreating the mash or feedstock with a high shear rotor stator device. In some embodiments, the rotor stator device has a gap between the surface of the rotor and the stator of between about 0.10 mm and 0.75 mm. In one embodiment, the mechanical pretreatment produces particles such that the majority of particles in the post-mechanically treated mash have a particle size between about 100 and 1600 microns, or between about 100 and 1000 microns. In one embodiment, the mechanical pretreatment produces particles such that the majority of particles have a particle size between about 100 and 1000 microns. For example, in some embodiments, the mechanical pretreatment produces particles such that greater than 85% of the particles by weight of the total non-dissolved solids in the post-mechanically treated mash have a particle size between about 100 and 1600 microns. In some embodiments, the mechanical pretreatment produces particles such that greater than 85% of the particles by weight of the total non-dissolved solids in the post-mechanically treated mash have a particle size between about 100 and 1000 microns.

In some embodiments, the additional enzymes are added to thin stillage and are dosed at a rate of between 0.05 to 0.75 ml of enzyme solution per liter of thin stillage.

In some embodiments, the viscosity of the thin stillage or syrup stream is reduced by at least 10%, 20%, 30%, 40% or 50%.

In some embodiments, the non-cellulosic sugars comprise starch. In some embodiments, the non-cellulosic sugars comprise starch derived from corn kernels, wheat, milo, sorghum, rice, maize, barley, sugar beets, or combinations thereof.

In some embodiments, the cellulosic sugars are derived from corn kernel fibers or corn kernel fibers plus other cellulosic feedstock comprising corn stover, paper or paper sludge, reprocessed paper or cardboard wastes, stalks, wood waste, or other low starch feedstock. In some embodiments, the other cellulosic feedstock has been preprocessed by a pretreatment step prior to adding the cellulase enzymes to the feedstock, or prior to fermenting the feedstock.

In some embodiments, the cellulosic sugars comprise less than 40%, less than 30%, less than 20%, or less than 10% by weight of the total hydrolysable polymeric sugars in the feedstock.

In another aspect, a method for reducing the viscosity of process streams during production of ethanol from biomass is provided, the method comprising:

a. fermenting a biomass comprising non-cellulosic sugars and cellulosic sugars to produce ethanol and a post-fermentation biomass;

b. generating process streams comprising whole stillage, thin stillage, concentrated thin stillage and/or syrup streams from the post fermentation biomass;

c. adding an additional enzyme or enzyme cocktail to at least one of the post fermentation process streams to reduce the viscosity of the syrup stream.

### BRIEF DESCRIPTION OF THE DRAWINGS

FIG. 1 shows the viscosity of the syrup can increase by a factor of three times normal values after low level dosing of cellulase enzymes into fermentation for two consecutive fermenter batches.

FIG. 2 provides an illustrative example of the inventive treatment and resulting viscosity reduction.

FIG. 3 shows the decreases in viscosity of thin stillage samples with various treatments.

FIG. 4 shows the decrease in viscosity using different VREs at enzyme loading levels of 0.5, 0.75, and 1.0 mg of enzyme solution per gram of thin stillage

### DEFINITIONS

Unless defined otherwise, all technical and scientific terms used herein have the same meaning as commonly understood by one of ordinary skill in the art to which this invention pertains. Although essentially any methods and materials similar to those described herein can be used in the practice or testing of the present invention, only exemplary methods and materials are described. For purposes of the present invention, the following terms are defined below.

The terms “a,” “an,” and “the” include plural referents, unless the context clearly indicates otherwise.

The term “about,” when modifying any amount, refers to the variation in that amount typically encountered by one of skill in the art, e.g., in processing biomass to produce ethanol. For example, the term “about” refers to the normal variation encountered in measurements for a given analytical technique, both within and between batches or samples. Thus, the term about can include variation of 1-10% of the measured value, such as 5% or 10% variation. The amounts disclosed herein include equivalents to those amounts, including amounts modified or not modified by the term “about.”

The term “biomass” or “biomass feedstock” refers to any material comprising lignocellulosic material. Lignocellulosic materials are composed of three main components: cellulose, hemicellulose, and lignin. Cellulose and hemicellulose contain carbohydrates including polysaccharides and oligosaccharides, and can be combined with additional components, such as protein and/or lipid. Examples of biomass include agricultural products such as grains, e.g., corn, corn kernels, wheat and barley; sugarcane; corn stover, corn cobs and other inedible waste parts of food plants; food waste; grasses such as switchgrass; and forestry biomass, such as wood, paper, board and waste wood products, as well as any solvent intermediates that contain any or any combination of the same in an aqueous solution phase.

The term “lignocellulosic” refers to material comprising both lignin and cellulose, and may also contain hemicellulose.

The term “cellulosic,” in reference to a material or composition, refers to a material comprising cellulose or comprising cellulose and hemicellulose.

The term “saccharification” refers to production of fermentable sugars from biomass or biomass feedstock. Saccharification can be accomplished by hydrolytic enzymes and/or auxiliary proteins, including, but not limited to, peroxidases, laccases, expansins and swollenins

The term “fermentable sugar” refers to a sugar that can be converted to ethanol or other products such as butanols, propanols, succinic acid, and isoprene, for example, during fermentation, for example during fermentation by yeast. For example, glucose is a fermentable sugar derived from hydrolysis of cellulose, whereas xylose, arabinose, mannose and galactose are fermentable sugars derived from hydrolysis of hemicellulose.

The term “non-cellulosic sugar” refers to a sugar derived from a non-cellulosic material, such as starch or inulin. The term “cellulosic sugar” refers to a sugar derived from a lignocellulosic and/or a cellulosic material. Some sugars, such as glucose, can be derived from both a non-cellulosic material such as alpha-glucan or a cellulosic material such as beta-glucan.

The term “simultaneous saccharification and fermentation” (SSF) refers to providing saccharification enzymes during the fermentation process. This is in contrast to the term “separate hydrolysis and fermentation” (SHF) steps.

The term “pretreatment” refers to treating the biomass with physical, chemical or biological means, or any combination thereof, to render the biomass more susceptible to hydrolysis, for example, by saccharification enzymes. Pretreatment can comprise treating the biomass at elevated pressures and/or elevated temperatures. Pretreatment can further comprise physically mixing and/or milling the biomass in order to reduce the size of the biomass particles and to disrupt the lignocellulosic structure. Devices that are useful for physical pretreatment of biomass include, e.g., a hammermill, shear mill, cavitation mill, or high shear rotor stator mill (e.g., a colloid mill). An exemplary colloid mill is the Cellunator™ (Edeniq, Inc., Visalia, Calif.). Reduction of particle size is described in, for example, WO2010/025171, which is incorporated by reference herein in its entirety.

The term “pretreated biomass” refers to biomass that has been subjected to pretreatment to render the biomass more susceptible to hydrolysis.

The term “elevated pressure,” in the context of a pretreatment step, refers to a pressure above atmospheric pressure (e.g., 1 atm at sea level) based on the elevation, for example at least 20, 30, 40, 50, 60, 70, 80, 90, 100, 110, 120, 130, 150, 200 or 250 psi or greater at sea level.

The term “elevated temperature,” in the context of a pretreatment step, refers to a temperature above ambient temperature, for example at least 100, 110, 120, 130, 140, 150, 160, 170, 180, 190, or 200 degrees C. or greater. When used in high pressure—high temperature (HPHT) pretreatment, the term includes temperatures sufficient to substantially increase the pressure in a closed system. For example, the temperature in a closed system can be increased such that the pressure is at least 100 psi or greater, such as 110, 120, 130, 150, 200 psi or greater.

The term “hydrolysis” refers to breaking the glycosidic bonds in polysaccharides to yield simple monomeric and/or oligomeric sugars. For example, hydrolysis of cellulose comprising beta-glucan produces the six carbon (C6) sugar glucose, whereas hydrolysis of hemicellulose produces the five carbon (C5) sugars xylose and arabinose. Hydrolysis can be accomplished by acid treatment or by enzymes such as cellulase, β-glucosidase, and xylanase. Examples of hydrolytic enzymes include cellulases and hemicellulases. Cellulase is a generic term for a multi-enzyme mixture or cocktail comprising exo-cellobiohydrolases, endoglucanases and β-glucosidases which work in combination to hydrolyze cellulose to cellobiose and glucose.

The terms “cellulase, cellulases, or cellulase cocktails” refer to any group of enzymes capable of hydrolyzing cellulose and hemicellulose or structural carbohydrates or fibers. The terms “hemicellulase, hemicellulases, or hemicellulase cocktail” refer to a group of enzymes capable of hydrolyzing only hemicellulose.

The term “viscosity reducing enzyme” or “viscosity reducing enzyme cocktail” refers to an enzyme mixture comprising xylanases, beta-glucosidases, arabinofuranosidases and/or other cellulases, for example, that is capable of reducing the viscosity of the stream being treated or a stream in the process that is downstream and generated from the treated stream. Representative viscosity reducing enzymes are shown in Table 1.

The term “co-fermentation” refers generally to fermenting sugars derived from starch and sugars derived from cellulose and/or hemicellulose in the same fermentation reaction.

### DETAILED DESCRIPTION OF THE INVENTION

The fiber content in the corn kernel represents about 6-12% of its dry mass. The corn kernel consists of 70 to 75% starch, 8-9% protein, 3.5-4% fat/oil, and the balance being fiber and ash. The fiber is about 30% lignin and the balance cellulose and hemi-cellulose. This balance can be further defined as 3.2% Glucan, 2.2% Xylan, 0.5% Galactan, 1.5% Arabinan, and 0.1% Mannan. By dosing commercial cellulase cocktails such as Accellerace TRIO from Dupont, or CTEC from Novozymes, into the fermentation mash, approximately 10 to 40% of the non-lignin fiber content is converted into shorter chain sugars and glucose (C6 sugar) or Xylose (C5 sugar). The glucose is consumed by the baseline yeasts in the ethanol plant and converted into cellulosic ethanol.

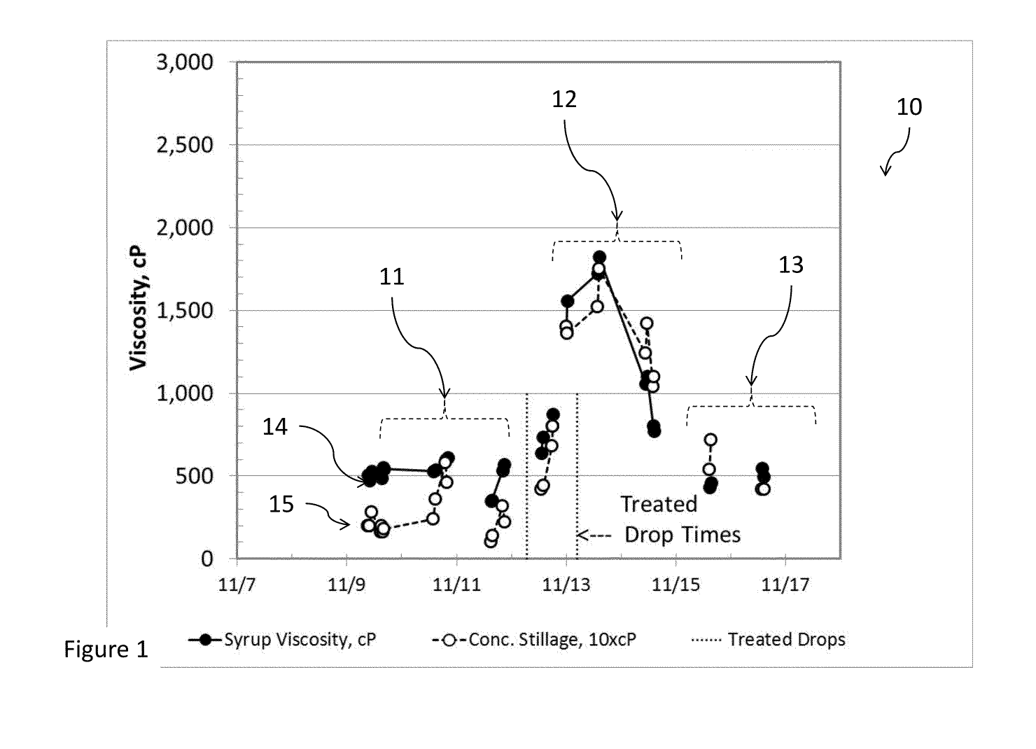

These commercially available cocktails are not specific to only the C6 sugars and in general attack bonds in much of the fiber structure and release other polymers and short chain sugar oligomers, some of which are soluble and some of which are not. The released soluble polymers include various Arabinoxylan polymers and related compounds which have relatively long side chains that can intertwine with each other and in so doing bond or cage a high percentage of the water. These characteristics result in increasing the viscosity of the thin stillage or centrate produced by the decanter centrifuges designed to extract most of the non-soluble, non-fermented solids. The thin stillage is typically concentrated into syrup from 10% solids up to about 30 to 40% solids in the evaporator trains, and at this point the viscosity can be a major issue, decreasing flow characteristics, coating walls, damaging pumps, plugging piping networks, etc. As illustrated in FIG. 1, the viscosity of the syrup can increase by a factor of three times normal values. FIG. 1 illustrates the viscosity of the syrup increases after a low level dosing of enzymes to two consecutive fermenter batches. Shown in the FIG. 1 (10) are the results for a typical Delta T style corn ethanol facility in which two consecutive fermentation batches were treated with cellulase enzymes in addition to mechanical pretreatment of the slurry. The viscosity of the concentrated thin stillage (15) as open data points and of the syrup (14) as solid data points are plotted as a function of time. The values of the concentrated thin stillage (15) have been increased by a factor of 10 to match the scale of the syrup data. The initial group of data (11) represents normal baseline characteristics as compared to the cellulase treated post fermentation drops. The treated post fermentation drops occurred at the two vertical dotted lines and the material had a typical delay as it passed through distillation, whole stillage, one stage of evaporation, and decanter centrifugation before further evaporation to become concentrated stillage and syrup. The treated group of data (12) represent the material dropped from the treated batches as the streams were processed into thin stillage after decanter centrifuge and then into concentrated thin stillage (15) and finally into syrup (14) in the evaporator trains. The final group of data (13) represents the stillage streams after the treated fermenter drops had passed through the system and replaced with untreated drop material. The relative viscosity of the treated material (12) is about 2 to 3 times the viscosity of the untreated material (11 and 13).

One potential solution to the problem of increased viscosity would be to eliminate all non-beta-glucan functionality in the commercial cellulase cocktails. However, this is not a practical solution because commercially available cocktails are generally designed for 100% cellulosic facilities in which both glucose and xylose are acceptable feed stocks for ethanol and other sugar to chemical processes.

As described herein, the present inventors have unexpectedly discovered another solution is to add to the commercially available cellulase cocktails an additional enzyme or enzyme cocktail that is capable of aggressively hydrolyzing soluble arabinoxylan and other pentosan-containing polymers and oligomers. By hydrolyzing the longer chain compounds into short chain oligomers or simple sugars the impact of the viscosity increase can be eliminated from the downstream evaporation processes. One embodiment of a secondary cocktail is a combination of xylanases, beta-glucosidases, and arabinofuranosidases. The additional enzyme or enzyme cocktail can be introduced at the fermentation stage, whole stillage stage, thin stillage stage, or evaporator stage depending on time and temperature activities of the specific enzymes. Other enzymes which might support or supplement the viscosity reduction are listed in Table 1 below. Alternate embodiments of this invention include any combination and any dosing amount of these various enzymes. These enzymes are useful for the reduction of the viscosity of the thin stillage, concentrated thin stillage, and or syrup as it is generated in the evaporator train.

TABLE 1
Potential Enzymes for Viscosity Reduction

| | |
|---|---|
| Xylanases | |
| Beta-glucosidases | |
| Arabinofuranosidases | |
| Non-starch carbohydrate- | |
| hydrolyzing enzymes | |
| Debranching enzymes | Breaks down glycogen |
| Hemicellulases | |
| Pentosanases | General term for a plurality |
| | of enzyme groups |
| Xylanolytic enzymes | |
| Exoxylanases | |
| Endoxylanases | |
| Glucanases | |
| Exoglucanases | |
| Endo-beta-1,4-xylanases | |
| Exo-beta-1,4-xylosidase | |
| Alpha-L-arabinofuranosidase | |
| Endo-alpha-1,5-arabinanase | |
| Glucuronidases | Act on starch |
| Alpha-glucuronidase | Act on starch |
| Mannanases | Break down Mannans |
| Endo-beta-1,4-mannanase | Break down Mannans |
| Exo-beta-1,4-mannosidase | Break down Mannans |
| Alpha-galactosidase | |
| Endo-galactanase | |
| Xylosidases | |
| Acetyl xylan esterases | |
| Glycosidases | Breaks down starch |
| Beta-1,4-glycanases | |
| Pectinases | Break down pectins |
| Polygalactoronases | Very similar to pectinase |
| Esterases | |
| Amylases | |
| Phytases | To breakdown phytic acid |
| Peroxidases | Used in waste water treatment |
| Laccases | |
| Glucose oxidases | |
| Oxidoreductases | Catalyze and transfer electrons |
| Lipases | Break down glycerol backbone in lipids |
| Lipolytic enzymes | Breaks down fats |
| Proteolytic enzymes | Cleaves long chain peptides |
| Proteases | Cleaves peptide bond in protein |

The materials and methods described herein can be used with virtually any starch-based and starch-based blended biomass. Examples include corn, wheat, barley, potato, rice, and sorghum, and any combination of which with other feedstocks. Examples of other feedstocks include, without limitation, sugar crops (e.g., sugarcane, energy cane, Jerusalem artichoke, or sugar beet), forage crops (e.g., grasses, alfalfa, or clover), and oilseed crops (e.g., soybean, sunflower, or safflower); wood products such as trees, shrubs, and wood residues (e.g., sawdust, bark or the like from forest clearings and mills); waste products such as municipal solid waste (MSW; e.g., paper, food and yard wastes, or wood), process waste and paper sludge; and aquatic plants such as algae, water weeds, water hyacinths, or reeds and rushes.

The materials and methods described herein can be used to produce any number of biofuels from starch-based feedstocks. Biofuels include, without limitation, alcohols such as ethanol, methanol, propanol, and butanol(s), solvents such as acetone, and blends thereof. Although ethanol may be the predominant biofuel referred to in the disclosure herein, such use of ‘ethanol’ is not meant to limit the present disclosure. The materials and methods described herein can be used to convert sugars using fermentation or SSF processes to alcohol(s) such as ethanol, methanol, propanol, butanol(s), solvents such as acetone, and blends thereof. Other fermentation of sugars can result in useful products such as lactic acids, succinic acids, acetic acids, glycerol, and various intermediate chemicals used as polymer precursors or product additives. The intent of this disclosure is not to be limited by the primary product of the fermentation process that is using a combination of cellulosic and non-cellulosic sugars.

In some embodiments, the feedstock for the conversion process is a biomass feedstock comprising easily hydrolysable polymeric sugars, such as starch, and a lower fraction of fiber or structural carbohydrates. An example is corn kernel biomass used in corn ethanol facilities. The corn kernel consists of 68 to 75% starch, 8-9% protein, 3-4% fat/oil, and the remaining material or balance being primarily fiber and ash. The starch component is easily converted into monomeric sugars and short chain sugar oligomers, with various enzymatic and thermal processes. The fiber content in the corn kernel represents about 10-12% of the dry mass of the biomass feedstock. The fiber is about 30% lignin and the remaining material or balance being primarily cellulose and hemi-cellulose. The specific composition split between cellulose and hemi-cellulose is dependent on the specific feedstock variety and growing conditions. For example, the fiber content in corn kernel fiber can be further defined as a fraction of the total dry mass of the kernel, or 3.2% is Glucan, or cellulose and hemi-cellulose comprises 2.2% as Xylan, 0.5% as Galactan, 1.5% as Arabinan, and 0.1% as Mannan, based on average corn flour composition obtained from a Northeastern Iowa ethanol plant during the months of October and November 2012.

Other starch based grains or biomass feedstock, which primarily contains starch, sugar, inulin, or other easily hydrolysable polymeric carbohydrates and a smaller fraction of structural carbohydrates, are applicable to various embodiments of this process. Non-limiting examples of these starch/sugar/inulin based biomass feedstock are milo, sorghum, rice, wheat, maize, barley, sugar beets, Jerusalem artichokes, and sugar cane, which can be used as feedstock. In some embodiments, the feedstock comprises mixtures of these grains or easily hydrolysable biomass and structural carbohydrate biomass feedstock consisting of primarily cellulose and hemi-cellulose. Examples of structural carbohydrate biomass are corn stover, energy cane bagasse, rice stalks, wheat stalks, sunflower stalks, and wood waste or reprocessed paper or cardboard wastes, but the scope of the embodiments are not limited by these examples. An example of a biomass feedstock mixture is 95% wt corn kernels and 5% wt corn stover or preprocessed corn stover, and other mixtures such as 90/10, or 80/20, or 70/30, or 60/40 kernels to stover respectively. The structural carbohydrate fraction may or may not be preprocessed prior to the mixture. Preprocessing of the structural carbohydrate can be any method of pretreatment including any combination of elevated temperature and pressure pretreatment designed to enhance the saccharification of the structural carbohydrates during the SSF process. In some embodiments, the sugars derived from the cellulosic feedstock comprise less than 40%, less than 30%, less than 20%, or less than 10% by weight of the total hydrolysable polymeric sugars in the feedstock.

In some embodiments, the corn kernels are dry milled into flour and mixed with water and a-amylases and glucoamylase (amyloglucosidase) to convert the starch into fermentable sugars. Commercially available a-amylases and glucoamylase enzyme cocktails include Distillase SSF+ (GA), Distillase SSF (GA), Spezyme (AA), Fuelzyme (AA), G-Zyme 480 (GA), Avantec (AA), Liquozyme (AA) and others available from suppliers such as Novozymes North America, DuPont (E. I. du Pont de Nemours and Company), and Verenium Corporation. Although each specific enzyme cocktail can comprise some cellulases, the cellulase activity of these representative cocktails are typically less than 10% or less than 5% of the targeted α-amylase and glucoamylase activities. Such activities can be manipulated in varying degrees by optimizing process parameters such as temperature, particle size, pH, and residence. Modifications of these cocktails to enhance or increase cellulase activities are feasible and considered equivalent to dosing cellulase(s) in the processes described herein. In some embodiments, the feedstock is contacted or treated with a-amylases and/or glucoamylase (amyloglucosidase) and a cellulase or cellulase cocktail.

Hydrolysis of the starch is defined as liquefaction which typically occurs between the slurry mix tanks and the fermentation tanks The dry milling of the corn kernels is typically achieved using a hammer mill that uses a screen to control the particle size of the flour being processed. If the screen size is small the hammer mill has a tendency to create a large number of very fine particles that impact the viscosity of downstream stillage streams. Energy consumption of the hammer mill is also increased with finer screen meshes and the thermal environment of the dry flour increases which can also cause retrograding of the starch matter, which prevents easy hydrolysis by conventional amylases. In one embodiment the hammer mill is combined with a wet milling rotor stator device, such as a colloid mill, which has the advantage of further reducing the starch particle size, disrupting the fiber structures, minimizing the generation of very fine particles, lowering energy consumption, minimizing thermal requirements, and creating a more homogeneous slurry mash stream for liquefaction. The rotor and stator provide parallel working surfaces.

The ethanol, or other biofuel, may then be generated by fermentation using yeast in a simultaneous saccharification and fermentation (SSF) setting, as found in U.S. Pat. No. 8,563,282B2, entitled MATERIALS AND METHODS FOR CONVERTING BIOMASS TO BIOFUEL, which is incorporated herein by reference in its entirety. The primary SSF can involve different commercial α-amylases and glucoamylase (amyloglucosidase) to convert the starch into fermentable sugars. The biomass feed stream or slurry feed prior to fermentation can comprise solids of 5% wt, 10% wt, 15% wt, 20% wt, 30% wt, 35% wt, 40% wt, or greater. In a typical dry mill corn ethanol plant, the feed slurry is in the 30 to 35% wt concentration range. The commercial enzymes used to convert starch may include other proprietary enzymes to enhance starch or easily hydrolysable polymeric sugar component conversion into fermentable sugars. Higher ethanol titers and greater throughput or production capacity are achievable with higher solids concentrations, but typical fermentation efficiencies will decrease with higher solids and the yield or production per dry mass of feedstock will decrease.

In some embodiments, a corn or other biomass powder generated by a hammer mill is mixed with fresh water and backset, to make for example a 30% solids mash that can be passed through a colloidal mill. The gap setting in the colloidal mill controls maximum particle size. The fluid pumped into the milling head chamber can be at ambient temperature or heated, sometimes in the range of 90° C. to 100° C. Passing through the colloidal mill, particles from the hammer mill, e.g., of 100 to 3000 microns in size, can be typically processed to a range of about 100 to about 500 microns, or about 100 to about 1000 microns, or about 100 to 1600 microns. In some embodiments, the particle size after treatment with the colloid mill is in the 100 to 500 micron range. In some embodiments, at least 85% or at least 95% by weight of the total particles have a particle size of about 100 to about 1000 microns. In some embodiments, a colloid mill is used as the only pretreatment step in a biomass to biofuel production process. In some embodiments, a wet milling rotor stator device is used to pretreat biomass in a biomass to biofuel production process together with at least one other method of pretreatment. In some embodiments the pretreatment processes include one or more of comminuting the biomass using a hammer mill and hydrolyzing the biomass using an enzyme or cocktail of enzymes.

In some embodiments, pretreatment includes the use of one or more enzymes to hydrolyze the biomass. The enzymes can be selected from alpha amylase, beta-amylase, glucanase, glucoamylase, cellulase, beta-glucanase, beta-glucosidase, hemicellulase, exo- and endo-xylanase, arabinofuranosidase, mannanase, beta-mannanase, endomannanase, galactosidase, galactomannanase, pectinase, debranching enzyme, pentosanase, xylosidase, glucoronidase, galactosidase, acetyl xylan esterase, polygalactoronase, phytase, glucose oxidase, lipase, protease, ligninase, peroxidase, manganese peroxidase, lignin peroxidase, laccase, cellobiohydrolase, cellobiase, and endoglucanase or mixtures thereof.

Wet milling rotor stator devices are available in various sizes and materials of construction. A person skilled in the art would be able to optimize the size and metallurgy for various biomass types. For example, two IKA model MK2000/50 rotor stator devices (IKA Works, Wilmington, N.C.) can be utilized in duplex stainless steel for a 50 MMGPY (million gallons per year) corn fermentation process, while a single IKA® Works, Inc. model MK2000/50 comprised of 304 stainless steel parts is all that is required for a 10-15 MMGPY sugar cane bagasse cellulosic process. In each instance, gap size is optimized for the various feedstock material input as well as various flow rate conditions. The rotor stator mill can be used to enable the resulting particle size distribution through the practice of adjusting the gap that physically separates the rotor and stator during operation. A relatively precise particle size distribution can be obtained from much larger biomass material using a rotor stator mill in contrast to alternative pretreatment techniques such as communication with a hammer mill. An appropriate gap size on the rotor stator mill can produce a highly uniform suspension of biomass, where the maximum particle size of the biomass is greatly reduced and significantly more uniform compared to using only the communication device. The gap size for the rotor stator mill or the physical separation of the rotor and stator during operation of the mill used in a corn ethanol plant can range from 0.10-0.75 millimeters, e.g., from 0.10-0.52 millimeters, e.g., from 0.20-0.52 millimeters, such that the majority of particles have a particle size in the range of about 100-1600 microns, or about 100-1000 microns, or about 100-800 microns. For example, in some embodiments, a gap setting of 0.10-0.20 is used for corn stover or other cellulosic biomass and a gap setting of 0.2-0.4 mm or 0.2-0.3 mm is used for grains including but not limited to corn kernels. The use of a wet rotor stator mill is to produce relatively precise, uniform particles sizes with high surface area and results in a greater percentage of starch, fiber and sugar oligomers being made available for enzymatic conversion than a hammer mill alone, leading to improved yield. The gap between the rotor and stator also minimizes the generation of additional fine suspended particles below 100 microns which is critical for maintaining rheology of downstream process streams such as whole stillage, thin stillage, concentrated thin stillage, and syrup or any mixture of these post fermentation process streams. The ability to minimize the fine suspended particles less than 100 microns also enhances the fermentation process by limiting the osmotic pressure on the active yeast cells in fermentation. All of these advantages are provided by using a wet milling rotor stator device for final stage particle size reduction before liquefaction in combination with an initial stage particle size or flour generation device such as but not limited to a hammer mill. The hammer mill screen size can be greater than a number 5 ( 5/64 inch openings), 6 ( 6/64 inch openings), or number 7 ( 7/64 inch openings), or number 8 ( 8/64 inch opening) size in a standard US dry mill ethanol plant and the larger the screen size the lower energy consumption and higher throughput capacity without thermal stress on the starch like material. Using a larger hammer mill screen and lower gap in rotor stator wet milling device provides more uniform particles sizes and fewer fine particles less than 100 microns. In one embodiment, a number 7 or greater hammer mill screen and a 0.2 to 0.3 mm gap between the rotor and stator is used.

Typically, as discussed above, the finer the biomass the better the attained yield with respect to gallons of biofuel per ton of biomass. However, a factor in the overall process is the recovery and management of residual solids after the biofuel has been removed. If a large fraction of the non-fermentable particles are less than 100 microns, then recovery of the residual solids after fermentation is more difficult with conventional centrifugation equipment. If a large fraction of non-fermentable particles are less than 100 microns, or if that fraction of non-fermentable particles less than 100 microns is increased by the action of the milling devices or process employed, then osmotic pressure on the yeast during fermentation is increased and the solids concentration of the fermentation mash must be decreased to offset this osmotic pressure on the yeast, and the throughput of the fermentation process is decreased. For cellulosic processes that utilize rice straw, sugar cane, energy cane and other materials (such as those described above) where state of the art filtration equipment can be installed, biomass particle size can be from 50-350 microns, typically from 75-150 microns.

With the pretreatment of the corn mash or slurry and liquefaction of the starch components the mash is passed to the fermentation for conversion of the sugars into a primary product such as ethanol. Since the fibers have been mechanically pretreated and disrupted by shear forces as they passed between the surface of the rotor and stator in the wet milling device, the fermentation mash can be further treated with cellulase and hemicellulase type enzymes or enzyme cocktails which can contain, for example, the functions of endoglucanase that hydrolyzes the middle of the cellulosic polymer, cellobiohydrolase I that hydrolyzes the reducing end of the cellulosic polymer, cellobiohydrolase II that hydrolyzes the non-reducing end of the cellulosic polymer, and the beta-glucosidase (BG) which converts cellobiose into glucose, xylose or other monomeric sugars that can be consumed by the yeast to make ethanol or other products.

Once the bio-organisms such as yeast have decreased the concentration of monomeric sugars in the fermentation broth, the inhibition of the cellulase by high sugar concentrations is decreased thereby supporting the hydrolysis of the cellulosic component during SSF. Commercially available cellulase cocktails include Accellerase® TRIO™ (DuPont, Wilmington, Del., USA). In the co-fermentation process described herein, cellulase and hemicellulase enzymes are dosed over a wide range of concentrations and feed rates depending on the properties of the enzymes and the fiber composition and the fermentation conditions. In some embodiments, the cellulase enzyme cocktail will be dosed at a rate of 0.01%-20%, or at a rate of 0.3%-12.5%, or at a rate of 0.6%-5% (%v/w) ml of enzyme solution relative to the mass in grams of beta-glucan content in the mash or fiber. This approach to dosing is dependent on the concentration of active enzymes in the solution. In other embodiments, the enzyme could be dosed at 0.01-20 mg of total protein per gram of corn kernel fiber beta-glucan, or at 0.3-12.5 mg of total protein per gram of corn kernel fiber beta-glucan, or at 0.6-5 mg of total protein per gram of beta-glucan present in the corn kernel fiber. In another embodiment, enzyme could be dosed at 2.5-5000 CMC Endoglucanase Units per gram of corn kernel fiber beta-glucan, or at 75-3000 CMC Endoglucanase Units per gram of corn kernel fiber beta-glucan, or at 150-1250 CMC Endoglucanase Units per gram of corn kernel fiber beta-glucan. In another embodiment, enzyme could be dosed at the rate of 3-6000 ABX Xylanase Units per gram of corn kernel fiber beta-glucan, or at 90-3600 ABX Xylanase Units per gram of corn kernel fiber beta-glucan, or at 225-1500 ABX Xylanase Units per gram of corn kernel fiber beta-glucan. In yet another embodiment, the enzyme could be dosed at a rate of 2-4000 pNPG Beta-Glucosidase Units per gram of corn kernel fiber beta-glucan, or at 60-2500 pNPG Beta-Glucosidase Units per gram of corn kernel fiber beta-glucan, or at 120-1000 pNPG Beta-Glucosidase Units per gram of corn kernel fiber beta-glucan. Other similar embodiments will be obvious to those skilled in the art. Alternatively, one can express this feed rate range as 0.01 to 20 mg of total protein per gram of beta-glucan, since commercially available cellulase enzyme has a density of about 1 gm/ml or ranging from 1.02 to 1.07 gm/ml. Commercial enzyme solutions can be relatively pure concentrations or whole broth concentrations in which the enzymes are mixed with growth media and thus are usually used as more dilute solutions.

Cellulase and hemicellulase enzyme activity is typically optimum at 40 to 60° C. while typical glucose fermenting yeasts provide optimum glucose conversion at less than 37° C., and preferably between 28° C. and 35° C., or between 31° C. and 34° C. Combining the yeast functionality within these temperature ranges with the cellulase functionality at otherwise non-optimum temperatures for the cellulase functionality is a surprising result of the process described herein. The cellulase cocktail can be added to the pre-fermentation mash at any point upstream or during fermentation at which the mash temperature is generally compatible with the cellulase cocktail enzyme stability. Cellulase enzymes that might be added early during the slurry process may be deactivated by high temperature zones between slurry and liquefaction. Typical, liquefaction temperatures of between 65 to 98° C. or between 75 to 90° C. can denature the enzyme and destroy its functionality downstream. Cellulase and hemicellulase activity is inhibited by high concentration of monomeric sugars such as glucose and xylose. One aspect of the process described herein is the addition of the cellulases and hemicellulases downstream of liquefaction due to the higher temperatures and after mash cooling and during fermentation. When the feedstock comprises an easily hydrolysable polymeric sugar comprising starch or inulin or others as the primary component, and the secondary component is a cellulosic material such as corn kernel fiber or stover or other, the process provides for the liquefaction of the primary component first and the addition of the cellulase enzymes designed to hydrolyze the secondary cellulosic component second. Some embodiments of the process include adding the cellulase and hemicellulase enzymes into the primary pre-fermentation mash after the mash has been cooled to below 70° C., or below 60° C., or below 55° C. where cellulase and hemicellulase functionality may be at or closer to optimum.

The liquefaction of the primary component often results in a post liquefied mash in which 20% to 100% hydrolysis of the primary polymeric sugar, or 20% to 60%, or 20% to 40% has been hydrolyzed to typical short chain sugars consisting of less than 6 to 8 monomeric sugar elements and a majority can be measured as DP4+, DP3, DP2, DP1 sugars by standard HPLC analysis (high performance liquid chromatography), where DP1 represents a monomeric sugar or monsaccharide or dextrose, DP2 represents a disaccharide or cellobiose or maltose, DP3 represents a trisaccharide or three unit sugar oligomer, and DP+4 represents longer chain sugar oligomers. The degree of hydrolysis of the soluble sugars can be defined as:

(1-(1-EqGlu/Sum(DPx))/(1−1.1111)),

where EqGlu is equal to [(DP4*1.08108)+(DP3*1.07143)+(DP2*1.05263)+(DP1)],

and Sum(DPx) is equal to (DP4+DP3+DP2+DP1).

The various constants are the (MW of Glucose)/(MW of DPx per sugar unit) and where 1.1111 is the (MW of Glucose/MW of Starch per sugar) or 180.2 gm/mole monomeric sugar divided by 162.1 gm/mole repeat unit in the starting polymer or oligomer. In typical corn mash liquefaction with corn solids of 28 to 32% wt the solids the initial equivalent glucose metric will be in the range of 25 to 35% w/v post liquefaction and the degree of hydrolysis will be in the 20% to 50% range depending on the effectiveness of the liquefaction process and enzymes and as a result the glucose concentration may be in the 6-12% w/v range early in fermentation.

Effective dosing of the cellulase and hemicellulase enzymes or enzyme cocktail (or cocktails) into fermentation is most effective when the glucose concentration has decreased to below 10% w/v or below 8% w/v or below 4% w/v or below 2% w/v and this will be typically be 8 to 30 hours after the start of the fermentation or preferable or between 8 to 20 hours, or between 8 to 12 hours after the start of fermentation. Glucose concentrations rapidly fall from 8% w/v to under 2% w/v between 10 hours and 30 hours after the start of fermentation. Similarly the DP4+concentration (representing longer chain oligomers) will be at 8 to 10% w/v at 10 hours and fall to under 2% w/v at 40 to 50 hours. When the cellulase enzyme cocktail is dosed into fermentation at about 10 hours an increase or bump in DP4+ concentration and the nominal decreasing profile can be observed between hours 15 and 30 indicating the release of cellulosic sugar oligomers into the fermentation mash or broth. If the cellulase cocktail is dosed into fermentation after the 30 hour point there is limited time for the cellulosic sugars to be hydrolyzed and released into the broth. Of course the fermentation time can be extended beyond the typical 40 to 70 hours cycle, but overall capacity or throughput of the plant will be decreased. Preferably the cellulosic enzyme cocktail is added to fermentation after 10 hours or after the glucose concentration has fallen below 6% w/v and such that there remains at least 30 hours of fermentation and SSF available before the batch is passed to downstream processes. The optimal time point for the cellulase enzyme cocktail may vary based on various fermentation parameters that deviate from refinery to refinery.

Recovery of ethanol can involve distillation to separate the ethanol from other components of the fermentation broth, and dehydration to remove residual water from the ethanol. The process typically used to recovery the product ethanol is distillation at elevated temperatures which has the secondary effect of denaturing or deactivating enzymes remaining in the post fermentation broth or mash. The post fermentation mash after ethanol removal is typically defined as whole stillage and comprises the residual starch, sugars and sugar oligomers as well as the non-fermentable components such as the fiber, protein, fats, yeast cells, etc. and the fermentation co-products such as glycerol, acetic acid, and lactic acid. This recovered post fermentation mash or whole stillage can be further processed or centrifuged to generate a liquid or fluid portion, often called thin stillage, and a solid portion of the whole stillage, often called wet grains. In some embodiments, the whole stillage is concentrated prior to downstream processes. In one embodiment, the whole stillage is not concentrated prior to downstream processes. The thin stillage can be further processed by various separation, evaporation, and/or concentration processes to eventually generate a syrup stream. The syrup can be mixed with the wet cake solids or sold as is. The mixed syrup and wet cake or modified wet cake can be sold as-is, partially dried, or dried to a moisture content of about 10%. The concentration of the syrup is limited by its rheology and the ability to process, move or convey the concentrated material to further downstream processes or distribution. Viscosity is one key metrics or characteristics of this rheology. Viscosity of the thin stillage and syrup streams are primarily a function of the longer organic molecules and the fine suspended particles. The longer chain organic molecules are sugar oligomers derived from partially hydrolyzed starch, cellulose and hemicellulose, and soluble protein and fat molecules. The fine suspended particles are fragments of the solid feedstock which remain after the starch is removed, fragments of non-hydrolyzed fibers, complexes of non-soluble proteins, and other components. There are many downstream processes which use the post fermentation stillage stream to recover additional high value co-products. U.S. Pat. No. 8,236,977 B2 entitled Recovery of Desired Co-Products from Fermentation Stillage Streams, and continuation U.S. patent applications Ser. Nos. 13/546,548 and 14/071,404 describe such downstream processes and are incorporated by reference herein in their entirety for all purposes.

Typical corn ethanol fermentation addresses the starch component of the feedstock and not the fiber or cellulosic portion, which passes through the process and remains primarily as suspended particles in the whole stillage and wet grains and as fine suspended particles in the centrate. Corn fiber to biofuel production can involve the use of a cellulase cocktail in combination with the use of a wet milling rotor stator device (such as but not limited to a colloidal mill) that can improve the utilization of the starch components and disrupt the fibrous components of the corn kernel to allow for both access and conversion of cellulosic components into fermentable sugar. As a result of this partial hydrolysis of the cellulosic compounds (e.g. cellulose and hemicellulose) the concentration of longer chain oligomers and quantity of fine suspended particles can increase or decrease in the whole stillage. As a result, the viscosity of the stillage streams and syrup in the downstream processes may increase from the mash treated with the cellulase cocktail due to rheology and compositional changes in the whole stillage. When the wet milling device is used independently of the cellulase cocktail, the concentration of the total dissolved solids can decrease in whole stillage due to the conversion of longer chain sugar oligomers from starch to glucose and ethanol or other products. Managing the quantity of the fine suspended, non-fermentable particles is one aspect of the combined dry milling (such as but not limited to hammer milling) and wet milling (such as but not limited to a colloid mill) process described herein.

When the cellulase cocktail is added to the fermentation reaction, some of the fine suspended cellulosic components of the mash are hydrolyzed and the various corn mash cell structures and walls are split. The composition of the post fermentation mash and whole stillage stream can be further modified by quantities such as the total solids, total suspended solids, fine suspended solids, and total dissolved solids. The splitting or lysing of the cell structures in the mash causes the bulk liquid within the cells to be released into the bulk post fermentation mash. This process has been described as dewatering (see Ana Beatriz Henriques, David B. Johnston, and Muthanna Al-Dahhanl, “Enhancing Water Removal from Whole Stillage by Enzyme Addition During Fermentation,” Cereal Chemistry September/October 2008, AACC International, Inc.; and Ana Beatriz Henriques, David B. Johnston, Andrew J. McAloon, and Milorad P. Dudukovic, “Reduction in energy usage during dry grind ethanol production by enhanced enzymatic dewatering of whole stillage: Plant trial, process model, and economic analysis,” Industrial Biotechnology August 2011, page 288). Dewatering of whole stillage was also described in U.S. Pat. No. 7,641,928 B2.

Management of the downstream processes in an ethanol facility is as important as preparing the mash for fermentation and the fermentation protocol. The downstream processes are focused at recovery of the primary product such as ethanol or other product, recovery of the process water needed for fermentation, and processing the residual solids as viable co-products such as dried distiller's grains, corn oil, organic acids, high concentrated syrup, and others. Initially, in an ethanol facility the post fermentation mash is passed to distillation for recovery of the product ethanol. The whole stillage can be initially concentrated or can be directly passed to a separations process, typically using a decanter centrifuge, to separate the bulk solids or the large suspended solids, cell fragments and non-lysed cells, non-hydrolyzed fibers as wet cake from the bulk liquid or centrate. The separation effectiveness of the decanter centrifuge can be adjusted to slightly tailor the separation characteristics. Because of the changes in the composition of the whole stillage the composition of the thin stillage will also change and these changes can cause an increase in the viscosity of the concentrated thin stillage and syrup that exits the evaporator process. Process problems can result from pumping and processing the concentrated thin stillage and syrup due to this viscosity. The viscosity may be lowered by treating the thin stillage stream, prior to evaporation, with a viscosity reducing enzyme (VRE) cocktail. Dosing of these VRE cocktails, which can be various mixtures of cellulase and hemicellulase enzymes added to the thin stillage stream, can reduce the ultimate viscosity of the syrup after evaporation and concentration. The VRE cocktail can consist of various specific enzyme activities comprising xylanases, beta-glucosidases, arabinofuranosidases and other cellulases and hemicellulases, individually or in mixtures thereof. The cocktails can also comprise non-starch carbohydrate-hydrolyzing enzymes selected from the group consisting of debranching enzymes, hemicellulases, pentosanases, xylanolytic enzymes, exoxylanases, endoxylanases, glucanases, exoglucanases, endo-beta-1,4-xylanases, exo-beta-1,4-xylosidase, alpha-L-rabinofuranosidase, endo-alpha-1,5-arabinanase, glucuronidases, alpha-glucuronidase, mannanases, endo-beta-1,4-mannanase, exo-beta-1,4-mannosidase, alpha-galactosidase, endo-galactanase, xylosidases, acetyl xylan esterases, glycosidases, beta-1,4-glycanases, pectinases, polygalactoronases, esterases, amylases, phytases, peroxidases, laccases, glucose oxidases, oxidoreductases, lipases, lipolytic enzyme, proteolytic enzymes, and proteases.

The enzyme compositions, specifically VREs, and methods described herein are useful for decreasing the resulting viscosity of the material present in the evaporator train when enzymatic treatments using cellulase and hemicellulase cocktails are used in or upstream of SSF to increase sugar conversion of the fermentation broth. The VRE cocktail can be the same or different than the cellulase cocktail used during fermentation, but it has been unexpectedly discovered that using the same cocktail supports the dual functions of hydrolyzing the cellulosic material in fermentation and providing the enzymatic activity in thin stillage to reduce the viscosity of the syrup as it is concentrated. The thin stillage temperature ranges from about 45° C. to about 80° C. in some ethanol facilities where the use of VREs was proven effective. Cellulases, hemicellulases, xylanases and related enzymes and enzyme cocktails have been used in the production of biofuels. This process demonstrates that similar cocktails can be used to decrease the viscosity of the thin stillage or evaporator concentrate in corn-to-ethanol refineries and used to enhance the co-fermentation of starch and cellulose in the SSF process of the primary fermentation.

In some embodiments, the viscosity of the process stream, such as the whole stillage stream, thin stillage stream, concentrated this stillage stream, or syrup stream, is reduced by at least 10%, 20%, 30%, 40%, 50% or more when the process stream is treated with a VRE or VRE cocktail as described herein. In some embodiments, the viscosity of the process stream, such as the whole stillage stream, thin stillage stream, concentrated this stillage stream, or syrup stream, is reduced by at least 10%, 20%, 30%, 40%, 50% or more when the post-fermentation process stream is treated with a VRE or VRE cocktail when compared to a method that does not treat the post-fermentation process stream with a VRE or VRE cocktail under substantially similar conditions.

Commercially available cocktails are not specific to only the glucose or C6 sugars, and in general attack bonds in much of the fiber structure and release other polymers and short chain sugar oligomers, some of which are soluble and some of which are not. The released soluble polymers or oligomers include various arabinoxylan polymers and related compounds which have reactive side chains that can intertwine with each other and in so doing sequester a high percentage of the water. These side chains can also interact with protein that is present in the ongoing fermentation broth, which can likewise interact with other protein-binding molecules to further sequester water molecules. Other viscosity-increasing mechanisms may play a role, such as further enzymatic hydrolysis or partial enzymatic hydrolysis of starch polymers that are not fully hydrolyzed into monomeric glucose. Such polymers and oligomers can further form interactions with both protein and reactive side-chains from cellulosic compounds that not only increase viscosity by staying insoluble, but also through further sequestering of water (and other) molecules. These characteristics result in increasing the viscosity of the thin stillage or centrate produced by the decanter centrifuges designed to extract most of the non-soluble, non-fermented solids. The thin stillage is typically concentrated into syrup from 6% to 10% solids up to about 30% to 40% solids in the evaporator trains, and at this point the viscosity can be a major issue, with impacts that can include decreasing flow characteristics, coating walls, damaging pumps, plugging piping networks, reducing effective heat transfer in the evaporators, etc.

Another solution is to combine a secondary enzyme or enzyme cocktail that is capable of aggressively hydrolyzing the soluble arabinoxylan polymers and non-beta-glucan sugar polymers/oligomers, such as hemicelluloses, with the cellulase cocktails (primarily designed to address the beta-glucan content) that are added to the fermentation mash downstream of liquefaction to aggressively hydrolyze the non-glucose or C5 sugar oligomers, into shorter chain sugars that have reduced impact on downstream process stream viscosity. By hydrolyzing the longer chain compounds into short chain oligomers or simple sugars the impact of the viscosity increase can be eliminated from concentrated stillage and the downstream evaporation processes. One embodiment of a secondary cocktail is a combination of xylanases, beta-glucosidases, amylases, β-glucanases, pectinases, exoglucanases (cellobiohydrolases, CBHs), endoglucanses, ligninases, β-mannanases, ferulic acid esterases, and arabinofuranosidases. The secondary enzyme or enzyme cocktail can be introduced at the fermentation stage, whole stillage stage, thin stillage stage, or evaporator stage depending on time and temperature activities of the specific enzymes. Other enzymes which can degrade the substrates shown below might also support or supplement the viscosity reduction. Alternate embodiments of this invention include any combination and any dosing amount of these various enzymes. These enzymes are useful for the reduction of the viscosity of the syrup as it is generated in the evaporator train. Substrates comprising filter paper, beech wood xylan, carboxy methyl cellulose (CMC), cellobiose, corn fiber gum, potato starch and corn starch have been used to assess enzyme cocktails and enzyme activities at process temperatures between 30-75° C. typical of thin stillage and thin stillage concentrate and produce the desired viscosity reduction of the concentrated thin stillage and or syrup.

In some embodiments, the VRE enzyme or additional enzyme cocktail is added into the feed stream leading to the whole stillage tank. In some embodiments, the VRE enzyme or additional enzyme cocktail is added to the centrate tank or directly into the thin stillage tank. In some embodiments, the VRE enzyme is added into the feed stream leading to the thin stillage tank. In some embodiments, the effective dose (ml of enzyme solution) is between a range of about 0.01 to 3.4 ml/liter of thin stillage, between about 0.05 to 2.5 ml/liter, or between about 0.05 to 0.75 ml/liter of thin stillage. Variations in the composition and concentration of the thin stillage and the desired level of viscosity reduction will affect the most cost effective dosing strategy. These illustrative dosing strategies are for an additional cellulase cocktail or VRE solution with about 8% to 12% solids and provided as a whole broth mixture with cellulase activity similar to the cocktails used for fermentation dosing, such as Accellerase® TRIO™ from DuPont.

Variations in the composition and concentration of the thin stillage and the desired level of viscosity reduction will affect the most cost effective dosing strategy. These illustrative dosing strategies are for a cellulase cocktail or VRE solution with about 8% to 12% solids and provided as a whole broth mixture with cellulase activity similar to the cocktails used for fermentation dosing. For example, in one embodiment, the thin stillage has a solids concentration of 11% wt and the VRE enzyme cocktail comprising about 2000 CMC Units/ml, at least about 3000 ABX Units/ml, and at least about 2000 pNPG units/ml.

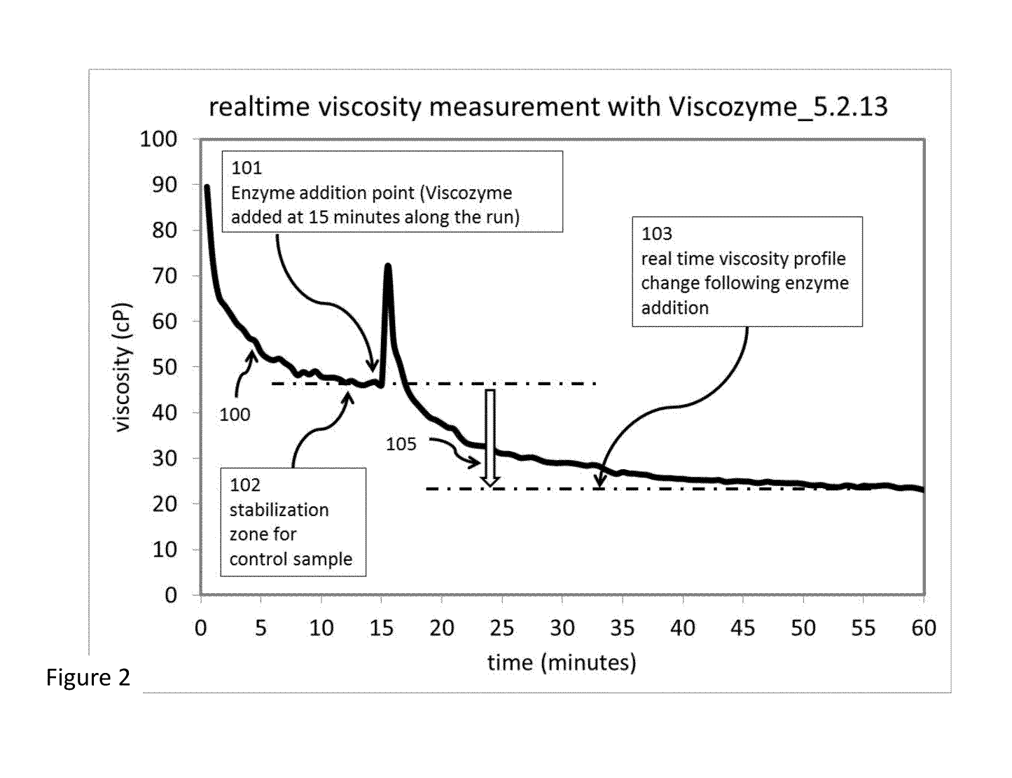

As can be seen from the viscosity versus time plot in FIG. 2, a syrup sample obtained from a commercial refinery was obtained and diluted with buffer, and then treated with an enzyme cocktail containing similar enzymes proposed and measured for viscosity with respect to time as shown (100). The viscosity versus time indicates that prior to the enzymatic treatment (102), the viscosity of the diluted sample is approximately 50 cP. The enzyme solution was added after 15 minutes (101) causing a momentary blip in the instrument readout. After treatment, the viscosity of the syrup rapidly declined between 15 and 20 minutes, and was reduced to roughly 22-25 cP at steady state at 60 minutes (103), thereby reducing the overall viscosity by 50%.

The evaporator concentrate is known to contain all of the solids, suspended and dissolved, that were not centrifuged into the wet cake portion after distillation and centrifugation. The fluid known as thin stillage, generated from centrifugation, is concentrated through a series of evaporators which can reach a total dry mass percent between 25-40%. The viscosity of the stream can vary, depending on the composition of the feedstock, ratio of dissolved solids to suspended solids, enzyme additives, and the target solids and temperature of the final syrup product. The VRE cocktail is believed to decrease the overall viscosity of the resulting concentrated stillage regardless of the upstream manipulations, but the relative magnitude is subject to each upstream process change and potential variation.

As measured close to process temperatures, between 70-95° C. in a late-stage evaporator, apparent viscosity can be decreased by up to 40% in two hours depending on the amount of the cellulase cocktail used. Typical viscosity for a commercial refinery in a late-stage evaporator is observed in the range of 300 to 2,000 cP and the downstream process and equipment typically are effective with viscosities of 300 to 1500 cP. The degree of viscosity reduction can be tailored via enzyme dose as well as enzyme cocktail composition.

Depending on operational conditions and feedstock composition, the enzyme cocktail may be used to reach basal viscosity levels that are desired for maintaining the standard process respective of each facility. By increasing both the reaction time of the enzyme with the substrates and/or the amount of enzyme usage per mass of substrate, the evaporator concentrate may be adjusted for the desired viscosity. Also due to the apparent dewatering effects on the centrate stream, the VREs may also enhance the efficiency of water recovery in the evaporator train.

In some embodiments, the enzyme cocktail is added into the thin stillage and/or thin stillage tank which are located prior to several, or all, evaporators in the series. The enzyme addition can happen intermittently and can be adjusted in loading to achieve the desired viscosity in the downstream product. The centrate stream which is typically divided into the backset stream and the thin stillage stream can have a pH between pH 3.0 to 6.5 and can have a temperature between 30° C. and 105° C. VRE dosing is preferred at a zone of the process where the temperature is between 35° C. and 70° C. Enzyme activities may fluctuate at different areas in each process, but as long as sufficient enzyme amounts are applied, viscosity reduction is possible in certain areas throughout the process. Viscosity reduction at the early stages of the evaporator train will have a relative reduction in viscosity as the fluid becomes more concentrated.

In some embodiments, the enzyme cocktail may be added directly into the evaporator train.

### EXAMPLES

The following examples are offered to illustrate, but not to limit the claimed invention.

### Example 1

This example demonstrates that representative cellulase and xylanase activities in commercial enzyme cocktails can be used to effectively reduce thin syrup viscosities in corn ethanol facilities that produce ethanol by co-fermentation of starch based glucose and cellulose based glucose.

Viscosity Reducing Enzymes and Specific Activities: A syrup sample, obtained from a commercial plant in which cellulases where introduced during fermentation at hour 10 to hydrolyze corn kernel fiber, was diluted with NaOAc pH 5.0 buffer and treated with 200 uL of either Accellerase TRIO or Accellerase 1500 (DuPont, Palo Alto, Calif.). Viscosity was measured using an R/S plus Rheometer with CC3-48 system (Brookfield) over the course of the reaction. As can be seen from the viscosity versus time plot in FIG. 2, a syrup sample obtained from a commercial refinery was obtained and diluted with buffer, and then treated with an enzyme cocktail and measured for viscosity with respect to time as shown (100). The viscosity versus time plot indicates that prior to the enzymatic treatment (102), the viscosity of the diluted sample is approximately 50 cP. The enzyme solution was added after 15 minutes (101) causing a momentary blip in the instrument readout. After treatment, the viscosity of the syrup rapidly declined between 15 and 20 minutes and reached a steady state of roughly 22-25 cP at 60 minutes (103), thereby reducing the overall viscosity by 50% (105). The reduction of total viscosity was roughly 50% in both cases. The publically available specifications for each cocktail are shown in TABLE 2. It is clear from this example that the activities of endo-glucanase, xylanase, and beta-glucosidase can be employed to reduce thin syrup viscosities.

TABLE 2
Specific Enzyme Activites of Accellerase
TRIO and Accellerase 1500

| Accellerase TRIO | Accelerase 1500 |
|---|---|
| 2000-2600 CMC Endoglucanase | 2200-2800 CMC Endoglucanase |
| Units/gm | Units/gm |
| >3000 ABX Xylanase Units/gm | 525-775 pNPG Beta-Glucosidase |
| | Units/gm |
| >2000 pNPG Beta-Glucosidase | |
| Units/gm | |

### Example 2

This example demonstrates that Viscosity Reducing Enzymes reduced the viscosity of thin stillage produced in Delta T style corn ethanol manufacturing facility that produce ethanol by co-fermentation of starch based glucose and cellulose based glucose.

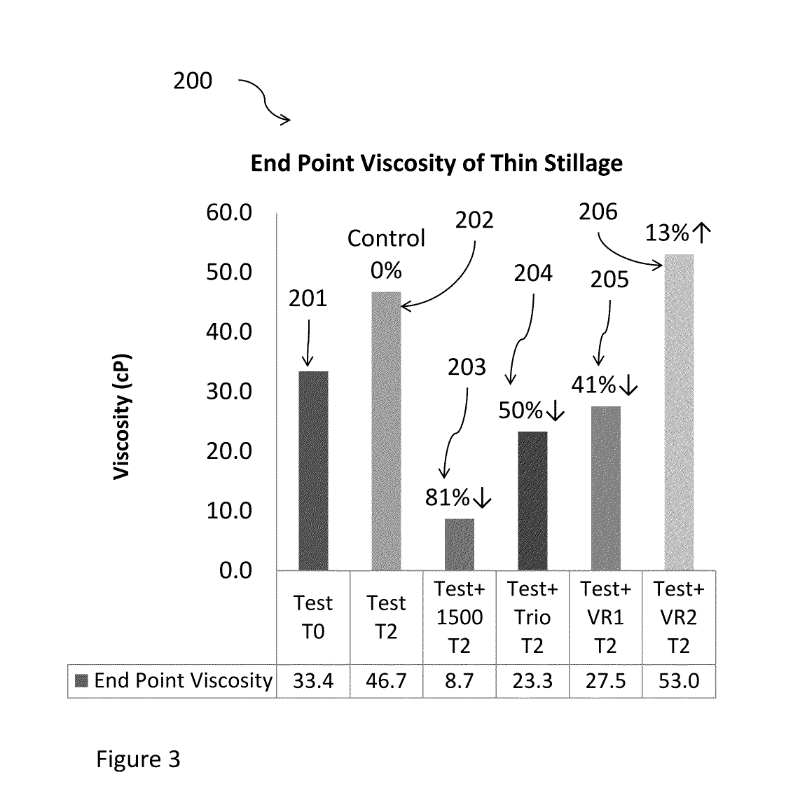

Viscosity Reducing Enzymes in thin stillage: Cellulase and hemicellulase enzymes at a dosing level of 63 gallons were added to an 800,000 gallon fermentation tank at hour 10 for a dosing of approximately 0.8% to 1.0% enzyme solution per gram of beta-glucan in the mash. Monitoring and timing the passage of the fermentation drop through distillation, first stage evaporation and centrifugation, samples of the thin stillage were obtained. This thin stillage was treated in the lab with four different enzyme cocktails obtained from various suppliers. The different enzyme cocktails used in this example are Accellerase 1500, Accellerase TRIO from DuPont, and two experimental blends labelled VR1 and VR2 from DuPont. Results from this experiment are shown in FIG. 3 (200). The sample was heated and maintained at 75° C. to simulate the conditions in the thin stillage tank and viscosity was measured at T=0 hours of reaching 75° C. (201) and after two hours of residence time (202) to establish a control viscosity. The different treatments were applied to the samples at a dose of 3.3 mg of solution per gram of thin stillage and viscosity was measured after two hours of residence time. The control is the Test T2 viscosity of about 45 cP and the percent decrease in viscosity using the different treatments are illustrated above each bar. Accellerase 1500 (203) resulted in the greatest decrease in viscosity with 81% reduction, and Accellerase® TRIO™ (204) resulted in a 50% decrease. The experimental VRE1 and VRE2 (bars 205 and 206) illustrated less effectiveness.

All viscosity measurements were performed with the R/S plus rheometer using the CC3-48 system.

### Example 3

This example demonstrates that VRE reduced the viscosity of thin stillage samples produced in a corn ethanol manufacturing facility using co-fermentation to produce ethanol.

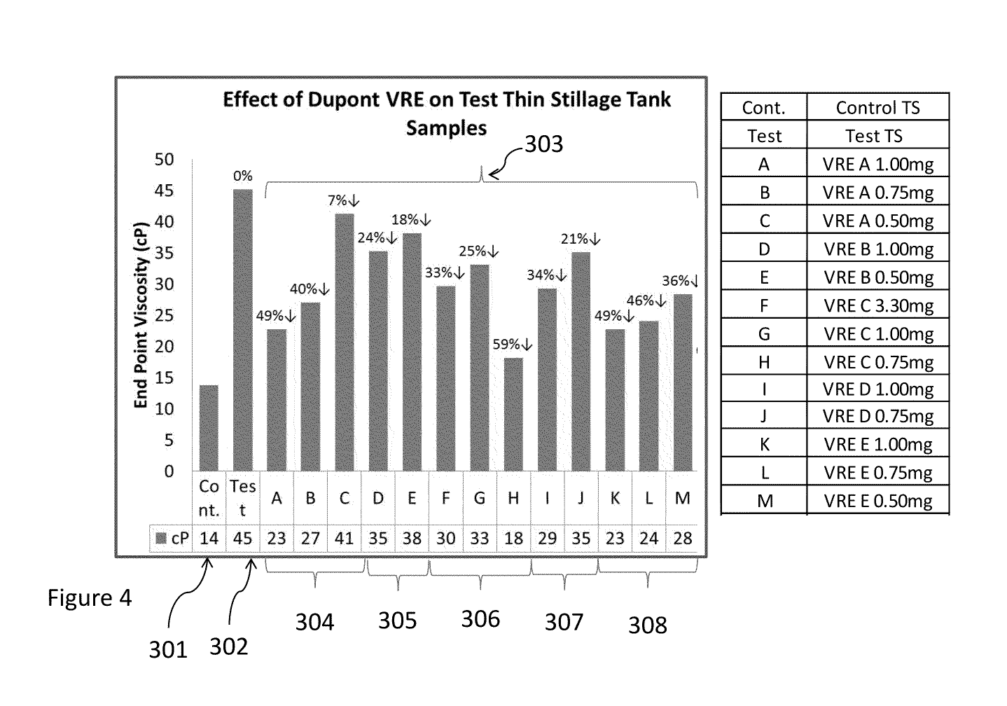

Varying VRE loading levels on thin stillage: Thin stillage was obtained from a commercial plant in which 63 gallons of a cellulase and hemicellulase enzyme cocktail was introduced into approximately 800,000 gallon tank during fermentation to hydrolyze corn kernel fiber. The thin stillage was treated with different enzyme cocktails at varying enzyme loading levels. The different enzymes tested are in this experiment are labeled as VRE A through VRE E. VRE A is TRIO and VRE B is Accellerase 1500 from DuPont. VRE C and D are the experimental VRE 1 and 2 from example 2 and VRE E is a third experimental cocktail. The thin stillage sample was handled and measured for viscosity using the same methods as Example 2, but the enzyme treatment was varied using mass loadings at 0.50, 0.75, and 1.0 mg of enzyme per gram of thin stillage as indicated in FIG. 4. In FIG. 4, the first two bars indicate two different thin stillage samples: Control TS is thin stillage that was generated without the inclusion of cellulases and hemicellulases in the fermentation broth (301), while the Test TS is thin stillage from the cellulase-included co-fermentation (302). For this example, the viscosity decreases (303) shown are compared to the Test TS treatment (302). After two hours of residence time, Accellerase 1500 (305) resulted in a viscosity decrease of 24 and 18% using enzyme loadings of 1.0 and 0.5 mg of enzyme per gram of thin stillage, respectively. Accellerase TRIO (304) resulted in a viscosity decrease of 49, 40, and 7% at enzyme loadings of 1.0, 0.75, and 0.5 mg of enzyme per gram of thin stillage, respectively. The results for the experimental VRE C, D, and E are shown in the remaining bars (306, 307, and 308).

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

What is claimed is:

1. A method for reducing the viscosity of process streams during production of ethanol from biomass, the method comprising:
a. adding cellulase enzymes into a mash or feedstock comprising a mixture of a non-cellulosic sugar and a cellulosic sugar to produce a fermentation mash;
b. fermenting the mash to produce ethanol and a post-fermentation biomass;
c. generating process streams comprising whole stillage, thin stillage, concentrated thin stillage and or syrup streams from the post fermentation biomass;
d. adding an additional enzyme or enzyme cocktail to at least one of the said post fermentation process stream to reduce the viscosity of the syrup stream.

2. The method of claim 1, wherein the additional enzyme comprises xylanases, beta-glucosidases, and/or arabinofuranosidases in any combination.

3. The method of claim 1, further comprising introducing the additional enzyme or enzyme cocktail to the whole stillage, thin stillage, and/or syrup or any combination thereof.

4. The method of claim 3, wherein the additional enzyme or enzyme cocktail is added to the thin stillage stream.

5. The method of claim 1, wherein the additional enzyme or enzyme cocktail comprises one or more of the following: debranching enzymes, hemicellulases, pentosanases, xylanolytic enzymes, exoxylanases, endoxylanases, glucanases, exoglucanases, endo-beta-1,4-xylanases, exo-beta-1,4-xylosidase, alpha-L-rabinofuranosidase, endo-alpha-1,5-arabinanase, glucuronidases, alpha-glucuronidase, mannanases, endo-beta-1,4-mannanase, exo-beta-1,4-mannosidase, alpha-galactosidase, endo-galactanase, xylosidases, acetyl xylan esterases, glycosidases, beta-1,4-glycanases, pectinases, polygalactoronases, esterases, amylases, phytases, peroxidases, laccases, glucose oxidases, oxidoreductases, lipases, lipolytic enzyme, proteolytic enzymes, and/or proteases.

6. The method of claim 1, wherein said cellulase enzymes are added to the fermentation tank.

7. The method of claim 1, further comprising mechanically pretreating the mash or feedstock with a high shear rotor stator device.

8. The method of claim 7, wherein the rotor stator device has a gap between the surface of the rotor and the stator of between 0.10 mm and 0.75 mm.

9. The method of claim 7, wherein the mechanical pretreatment produces particles such that the majority of particles have a particle size between about 100 and 1000 microns.

10. The method of claim 9, wherein the majority is greater than 85% by weight of the total non-dissolved solids in the mash.

11. The method of claim 4, wherein the additional enzymes are dosed at a rate of between 0.05 to 0.75 ml of enzyme solution per liter of thin stillage.

12. The method of claim 1, wherein the viscosity of the thin stillage or syrup stream is reduced by at least 10%.

13. The method of claim 1, wherein the non-cellulosic sugars comprise starch derived from corn kernels, wheat, milo, sorghum, rice, maize, barley, sugar beets, or combinations thereof.

14. The method of claim 1, wherein the cellulosic sugars are derived from corn kernel fibers or corn kernel fibers plus other cellulosic feedstock comprising corn stover, paper or paper sludge, reprocessed paper or cardboard wastes, stalks, wood waste, or other low starch feedstock.

15. The method of claim 14, wherein the cellulosic sugars comprise less than 40% by weight of the total hydrolysable polymeric sugars in the feedstock.

16. The method of claim 14, wherein the other cellulosic feedstock has been preprocessed by a pretreatment step prior to adding the cellulase enzymes to the feedstock.

17. A method for reducing the viscosity of process streams during production of ethanol from biomass, the method comprising:
a. fermenting a biomass comprising non-cellulosic sugars and cellulosic sugars to produce ethanol and a post-fermentation biomass;
b. generating process streams comprising whole stillage, thin stillage, concentrated thin stillage and/or syrup streams from the post fermentation biomass;
c. adding an additional enzyme or enzyme cocktail to at least one of the post fermentation process streams to reduce the viscosity of the syrup stream.
